(19) United States
(12) Patent Application Publication
(10) Pub. No.: US 2020/0221961 A1
(43) Pub. Date: Jul. 16, 2020

(54) DISPLAY CONTROL DEVICE AND RECORDING MEDIUM OF PROGRAM

(71) Applicants: OMRON HEALTHCARE Co., Ltd., Kyoto (JP); OMRON Corporation, Kyoto (JP)

(72) Inventors: Daisuke ISHIHARA, Kyoto (JP); Yasuhiro KAWABATA, Kyoto (JP)

(21) Appl. No.: 16/812,464

(22) Filed: Mar. 9, 2020

Related U.S. Application Data
(63) Continuation of application No. PCT/JP2018/032903, filed on Sep. 5, 2018.

(30) Foreign Application Priority Data
Sep. 12, 2017 (JP) 2017-175113

(51) Int. Cl. A61B 5/021; A61B 5/00

(52) U.S. Cl. CPC A61B 5/02116; A61B 5/681; A61B 5/743; A61B 5/02125

(57) ABSTRACT

A display control device of the present disclosure is configured to present information for positioning a pulse wave sensor relative to a measurement site. A measurement device includes a belt wrapped around and attached to a measurement site for measuring a pulse transit time, a sensor unit provided on an inner peripheral surface of the belt that i toward the measurement site when the belt is attached, and a display provided on an outer peripheral surface of the belt opposite to the inner peripheral surface. The display is provided on the outer peripheral surface in a site configured to face a site where the sensor unit is positioned when the belt is attached, and the sensor unit includes a first pulse wave sensor and a second pulse wave sensor disposed spaced apart from each other in a width direction of the belt. First indicator information indicating a magnitude of a first pulse wave amplitude indicated by an output of the first pulse wave sensor, and second indicator information indicating a magnitude of a second pulse wave amplitude indicated by an output of the second pulse wave sensor are displayed on the display in positions respectively corresponding to the first pulse wave sensor and the second pulse wave sensor.

### CROSS-REFERENCE TO RELATED APPLICATIONS

This is a continuation of International Application PCT/JP2018/032903, with an international filing date of Sep. 5, 2018, and also JP 2017-175113 with a filing date of Sep. 12, 2017, filed by applicant, the disclosure of which is hereby incorporated by reference in its entirety.

### TECHNICAL FIELD

The present disclosure relates to a display control device and a recording medium of a program, and particularly relates to a display control device and a recording medium of a program for pulse wave information.

### BACKGROUND ART

Methods for positioning a pulse wave detection sensor on an artery to detect a pulse wave have been proposed. For example, Patent Document 1 discloses displaying a magnitude of a pressure pulse wave detected by pressure detection elements arranged in a single row, on a display. Further, Patent Document 2 discloses a configuration in which a position of a pulse wave detection circuit is adjusted while viewing a display of an amplitude value of a detected pulse wave.

### CITATION LIST

### Patent Literature

Patent Document 1: JP 2004-222814 A

Patent Document 2: WO 98/51025

Summary of Disclosure

### Technical Problem

In the related art, methods for estimating (measuring) blood pressure from a transit time of a pulse wave propagating through an artery (pulse transit time; PTT) are known. PTT is found from a time difference between the detection of peaks (maximums) of pulse wave amplitudes at two different points on the artery where pulse wave signals are detected by pulse wave sensors at each of the points. Thus, in order to achieve high blood pressure measurement accuracy, it is desirable to present information for reliably positioning the pulse wave sensors on the artery. However, in Patent Document 1 and Patent Document 2, information for positioning the pulse wave sensors when measuring the pulse transit time is not presented.

An object of an aspect of the present disclosure is to provide a display control device and a recording medium of a program configured to present information for positioning pulse wave sensors relative to measurement sites when measuring a pulse transit time.

### Solution to Problem

According to an aspect of this disclosure, provided is a display control device included in a measurement device. The measurement device includes a belt wrapped around and attached to a measurement site for measuring a pulse transit time, a sensor unit provided on an inner peripheral surface of the belt that is toward the measurement site when the belt is attached, and a display provided on an outer peripheral surface of the belt opposite to the inner peripheral surface.

The display is provided on the outer peripheral surface in a site configured to face a site where the sensor unit is positioned when the belt is attached, and the sensor unit includes a first pulse wave sensor and a second pulse wave sensor disposed spaced apart from each other in a width direction of the belt.

The display control device is configured to display first indicator information indicating a magnitude of a first pulse wave amplitude indicated by an output of the first pulse wave sensor, and second indicator information indicating a magnitude of a second pulse wave amplitude indicated by an output of the second pulse wave sensor, in positions on the display respectively corresponding to the first pulse wave sensor and the second pulse wave sensor disposed spaced apart.

Preferably, the display control device is further configured to display guidance information on the display for adjusting a relative positional relationship between the sensor unit and the measurement site, in accordance with the magnitude of the first pulse wave amplitude and the magnitude of the second pulse wave amplitude.

Preferably, the display control device is further configured to display the guidance information on a same screen of the display together with the first indicator information and the second indicator information.

Preferably, the guidance information includes information providing guidance related to a direction of movement for moving a position of the sensor unit relative to the measurement site.

Preferably, when the magnitude of the first pulse wave amplitude or the magnitude of the second pulse wave amplitude does not indicate a predetermined magnitude, the guidance information displays information providing guidance related to the direction of movement.

Preferably, when the magnitude of the first pulse wave amplitude and the magnitude of the second pulse wave amplitude indicate the predetermined magnitude, the guidance information includes information providing guidance related to securing a position of the sensor unit.

Preferably, when the magnitude of the first pulse wave amplitude and the magnitude of the second pulse wave amplitude indicate the predetermined magnitude, the guidance information includes information providing guidance related to securing the position of the sensor unit instead of information providing guidance related to the direction of movement.

Preferably, the information indicating a magnitude of a pulse wave amplitude corresponding to each of the first pulse wave amplitude and the second pulse wave amplitude has different display modes for when the magnitude of the pulse wave amplitude indicates the predetermined magnitude and when the magnitude of the pulse wave amplitude does not indicate the predetermined magnitude.

Preferably, the guidance information includes information for evaluating a state of the attachment relative to the measurement site, and the information for evaluating indicates different evaluations when the magnitude of the first pulse wave amplitude and the magnitude of the second pulse wave amplitude indicate the predetermined magnitude, and when the magnitude of the first pulse wave amplitude or the magnitude of the second pulse wave amplitude does not indicate the predetermined magnitude.

Preferably, when the magnitude of the first pulse wave amplitude or the magnitude of the second pulse wave amplitude does not indicate the predetermined magnitude, the guidance information includes information prompting the wrapping and the attaching again.

Preferably, the measurement device further includes a communication unit configured to communicate with an information processing device that is external and includes a displaying section, and is configured to transmit the guidance information to the information processing device via the communication unit to cause the displaying section to display the guidance information.

Preferably, the pulse transit time is calculated from the magnitude of the first pulse wave amplitude and the magnitude of the second pulse wave amplitude, and the measurement device is configured to calculate blood pressure on the basis of the pulse transit time.

Preferably, the display control device is further configured to display information for evaluating a state of the attachment relative to the measurement site in association with information for evaluating the blood pressure thus calculated, and the guidance information includes the information for evaluating a state of the attachment.

Preferably, the display control device is further configured to cause, while the guidance information is displayed, upon changing the magnitude of the first pulse wave amplitude or the magnitude of the second pulse wave amplitude, the guidance information to include information providing notification of the change.

According to another aspect of this disclosure, provided is a recording medium of a program for causing a computer to execute a display control method of a device. This device includes a belt wrapped around and attached to a measurement site for measuring a pulse transit time, a sensor unit provided on an inner peripheral surface of the belt that is toward the measurement site when the belt is attached, and a display provided on an outer peripheral surface of the belt opposite to the inner peripheral surface. The display is provided on the outer peripheral surface in a site configured to face a site where the sensor unit is positioned when the belt is attached, and the sensor unit includes a first pulse wave sensor and a second pulse wave sensor disposed spaced apart from each other in a width direction of the belt. The display control method displays the first indicator information and the second indicator information in positions respectively corresponding to the first pulse wave sensor and the second pulse wave sensor disposed spaced apart.

### Advantageous Effects of Disclosure

According to the present disclosure, information for positioning a pulse wave sensor may be presented when measuring a pulse transit time.

### BRIEF DESCRIPTION OF DRAWINGS

FIG. 1 is an appearance perspective view of a blood pressure monitor 1 according to a first embodiment.

FIG. 2 is a diagram illustrating a state in which the blood pressure monitor 1 according to the first embodiment is attached to a wrist 90 (left).

FIG. 3 is a diagram illustrating a planar layout of an electrode group for impedance measurement with the blood pressure monitor 1 in FIG. 1 attached to the wrist 90 (left).

FIG. 4 is a diagram illustrating a block configuration of a control system of the blood pressure monitor 1 according to the first embodiment.

FIGS. 5A and 5B are schematic diagrams for explaining a blood pressure measurement based on a pulse transit time according to the first embodiment.

FIG. 6 is a schematic cross-sectional view of the blood pressure monitor 1 attached to the wrist 90, in a longitudinal direction of the wrist, when blood pressure is measured by an oscillometric method according to the first embodiment.

FIGS. 7A to 7E are diagrams for explaining determination of an attachment state of a sensor unit according to the first embodiment.

FIG. 8 is a diagram schematically illustrating a configuration of a function for outputting guidance information in association with a blood pressure measurement function according to the first embodiment.

FIG. 9 is a flowchart illustrating processing of blood pressure measurement based on output of the guidance information and the pulse transit time according to the first embodiment.

FIGS. 10A to 10C are diagrams illustrating another display example of the guidance information according to the first embodiment.

FIGS. 11A to 11C are diagrams illustrating another display example of the guidance information according to the first embodiment.

FIGS. 12A to 12C are diagrams illustrating another display example of the guidance information according to the first embodiment.

FIG. 13 is a diagram illustrating a storage example of a measurement result according to the first embodiment.

FIGS. 14A and 14B are diagrams illustrating another display example according to the first embodiment.

FIGS. 15A to 15C are diagrams illustrating yet another display example according to the first embodiment.

FIG. 16 is a diagram illustrating yet another display example according to the first embodiment.

FIG. 17 is a diagram illustrating yet another display example according to the first embodiment.

FIG. 18 is a diagram illustrating a schematic configuration of a system according to a second embodiment.

### DESCRIPTION OF EMBODIMENTS

Embodiments of the present disclosure will be described below with reference to the drawings. In the following description, like components are given like numerals. Names and functions thereof are also the same. Thus, the detailed description of such components is not repeated.

Below, an example of a blood pressure monitor that is a wearable terminal as a device for measuring a pulse transit time (hereinafter referred to as PTT), and a case in which a “display control device” is mounted in the blood pressure monitor will be described. However, the device in which the “display control device” is mounted may be a device including a sensor that detects a pulse wave signal and a processing device configured to process a signal detected by the sensor, and is not limited to the blood pressure monitor. Further, the blood pressure monitor is not limited to a wearable terminal.

### First Embodiment

### Configuration of Blood Pressure Monitor

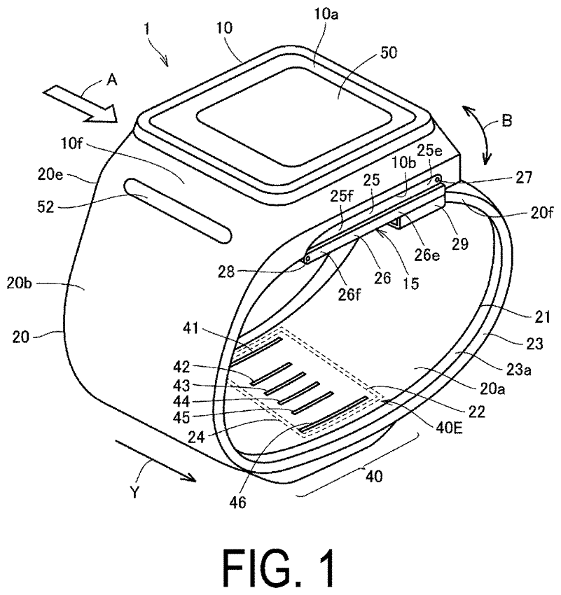

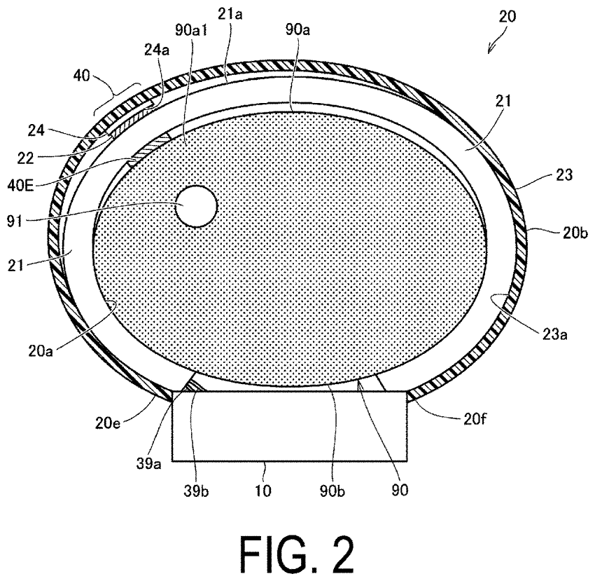

FIG. 1 is an appearance perspective view of a blood pressure monitor 1 according to a first embodiment. FIG. 2 is a diagram schematically illustrating a cross section of the blood pressure monitor 1 according to the first embodiment attached to the wrist 90 (left) (hereinafter referred to as “attachment state”), orthogonal to a longitudinal direction of the wrist 90. In the present embodiment, the wrist 90 (left) serves as a measurement site. Note that the “measurement site” need only be a site through which an artery passes, and is not limited to the wrist. The measurement site may be, for example, a right wrist, an upper arm, an ankle, a lower limb such as an upper thigh, or the like.

With reference to FIG. 1 and FIG. 2, a belt 20 is a band-like member. The belt 20 is positioned so that a longitudinal direction thereof corresponds to the wrist 90 in a circumferential direction, and is slidably wrapped and attached in an attachment state. A dimension of the belt 20 in a width direction Y (width dimension) is approximately 30 mm, for example. The belt 20 includes a band 23 and a compression cuff 21. The band 23 includes an inner peripheral surface 23a, which is a surface on the measurement site side, and an outer peripheral surface 20b, which is a surface on a side opposite to the inner peripheral surface 23a. In the first embodiment, when the belt 20 is wrapped around and attached to the measurement site, the state of the blood pressure monitor 1 is the “attachment state.” Further, “attached” indicates that this “attachment state” is ongoing.

The compression cuff 21 is attached along the inner peripheral surface 23a of the band 23 and includes an inner peripheral surface 20a that comes into contact with the wrist 90 (refer to FIG. 2). The compression cuff 21 is configured as a fluid bag with two stretchable polyurethane sheets facing each other in a thickness direction and edges thereof welded. In the present embodiment, the fluid bag of the compression cuff 21 may be a bag-like member capable of accommodating a fluid. The compression cuff 21 expands when fluid is supplied and the measurement site is pressurized in association with the expansion. Further, when the fluid is discharged, the compression cuff 21 contracts and the pressurized state of the measurement site is resolved.

A body 10 is integrally provided with one end portion 20e of the belt 20. Note that the belt 20 and the body 10 may be formed separately, and the body 10 may be integrally attached to the belt 20 via an engagement member (a hinge, for example). In the present embodiment, a site where the body 10 is disposed corresponds to a back side surface (surface on an back side of the hand) 90b of the wrist 90 in the attachment state (refer to FIG. 2). In FIG. 2, a radial artery 91 is illustrated passing through an area near a palm side surface (surface on a flat side of the hand) 90a in the wrist 90.

As illustrated in FIG. 1, the body 10 has a three-dimensional shape having a thickness in a direction orthogonal to the outer peripheral surface 20b of the belt 20. The body 10 is compact in size and formed to a thin thickness so as not to interfere with daily activity of a user. The body 10 has a truncated quadrangular pyramid profile protruding outwardly from the belt 20.

A display 50 is provided on a top surface (a surface farthest from the measurement site) 10a of the body 10. An operating section 52 for inputting an instruction from the user is provided along a side surface 10f (side surface on a left front side in FIG. 1) of the body 10.

A sensor unit 40 is provided on the inner peripheral surface 20a (that is, the inner peripheral surface 20a of the compression cuff 21) of the belt 20 in a site between the one end portion 20e and the other end portion 20f of the belt 20. The sensor unit 40 includes a function for detecting a pulse wave using an impedance measurement function.

An electrode group 40E is disposed on the inner peripheral surface 20a of the site where the sensor unit 40 is disposed. The electrode group 40E includes six electrodes 41 to 46 having a plate-shape (or sheet-shape) and spaced apart from each other in the width direction Y of the belt 20. The site where the electrode group 40E is disposed corresponds to the radial artery 91 of the wrist 90 in the attachment state.

A solid material 22 may be disposed on the outer peripheral surface 21a in a position corresponding to the electrode group 40E. A pressing cuff 24 is disposed on an outer peripheral side of the solid material 22. The pressing cuff 24 is an expandable member that locally suppresses a region corresponding to the electrode group 40E in the circumferential direction of the compression cuff 21. The pressing cuff 24 is disposed on the inner peripheral surface 23a of the band 23 constituting the belt 20 (refer to FIG. 2). The band 23 is formed from a plastic material flexible in the thickness direction and non-stretchable in the circumferential direction (longitudinal direction).

The pressing cuff 24 is a fluid bag that expands and contracts in the thickness direction of the belt 20, is in a pressurized state by the supply of fluid, and is in a non-pressurized state by the discharge of the fluid. The pressing cuff 24 is configured as, for example, a fluid bag with two stretchable polyurethane sheets facing each other in the thickness direction and the edges thereof welded.

The solid material 22 is disposed on the inner peripheral surface 24a of the pressing cuff 24 in a position corresponding to the electrode group 40E. The solid material 22 is constituted by, for example, a resin (polypropylene, for example) having a plate-like shape and a thickness of about from 1 to 2 mm. In the present embodiment, the belt 20, the pressing cuff 24, and the solid material 22 are used as a pressing portion.

As illustrated in FIG. 1, a bottom surface (surface closest to the measurement site) 10b of the body 10 and the end portion 20f of the belt 20 are connected by a tri-fold buckle 15 (hereinafter also simply referred to as “buckle 15”).

The buckle 15 includes a plate-like member 25 disposed on an outer peripheral side and a plate-like member 26 disposed on an inner peripheral side. One end portion 25e of the plate-like member 25 is attached in a freely rotatable manner to the body 10 via a connecting rod 27 extending in the width direction Y. The other end portion 25f of the plate-like member 25 is attached in a freely rotatable manner to one end portion 26e of the plate-like member 26 via a connecting rod 28 extending in the width direction Y. The other end portion 26f of the plate-like member 26 is fixed in the vicinity of the end portion 20f of the belt 20 by a fixing portion 29.

An attachment position of the fixing portion 29 is variably set in advance in the circumferential direction of the belt 20 in accordance with a circumferential length of the wrist 90 of the user. As a result, the blood pressure monitor 1 (belt 20) is configured to be substantially annular as a whole, and the bottom surface 10b of the body 10 and the end portion 20f of the belt 20 are configured to be openable and closeable in an arrow B direction in FIG. 1 by the buckle 15.

When the user attaches the blood pressure monitor 1 to the wrist 90, the user opens the buckle 15 and, with the diameter of the looped belt 20 made large, passes the left hand through the belt 20 from the direction indicated by the arrow A in FIG. 1. Next, as illustrated in FIG. 2, the user slides an angular position of the belt 20 around the wrist 90 and the like to adjust the position, and moves the sensor unit 40, positioning the sensor unit 40 on the radial artery 91. As a result, the electrode group 40E of the sensor unit 40 comes into contact with a portion 90a1 of the palm side surface 90a of the wrist 90 corresponding to the radial artery 91. In this state, the user closes and secures the buckle 15. Thus, the user wraps and attaches the blood pressure monitor 1 (belt 20) to the wrist 90.

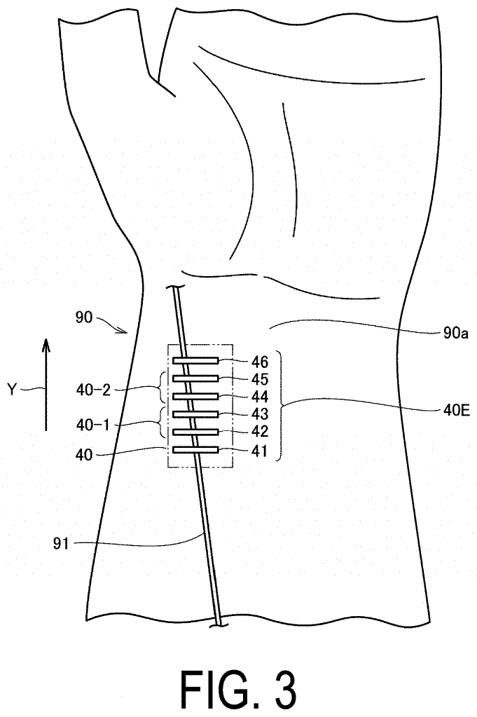

FIG. 3 is a diagram illustrating a planar layout of an electrode group for impedance measurement with the blood pressure monitor 1 according to the first embodiment attached to the wrist 90. With reference to FIG. 3, in the attachment state, the electrode group 40E of the sensor unit 40 is aligned in the longitudinal direction of the wrist in correspondence with the radial artery 91 of the wrist 90 (left). The electrode group 40E includes the current electrode pair 41, 46 for current flow disposed on both sides in the width direction Y, and the detection electrode pair 42, 43 as well as the detection electrode pair 44, 45 disposed between the current electrode pair 41, 46. A first pulse wave sensor 40-1 includes the detection electrode pair 42, 43 and a second pulse wave sensor 40-2 includes the detection electrode pair 44, 45.

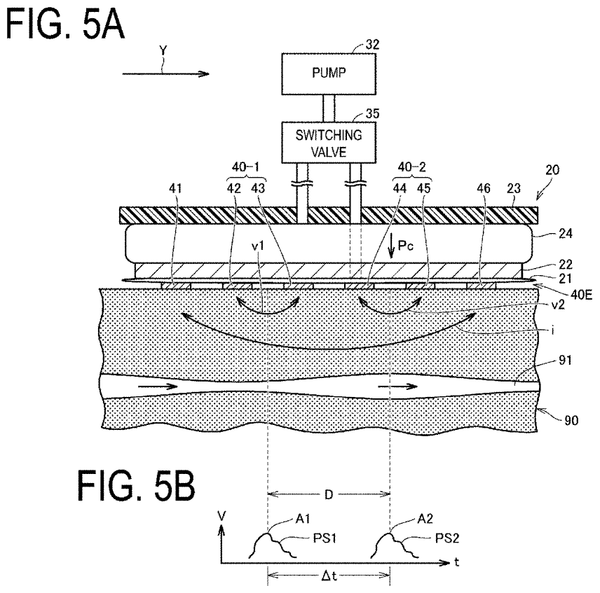

The detection electrode pair 44, 45 is disposed in correspondence with a portion downstream of the blood flow of the radial artery 91 relative to the detection electrode pair 42, 43. In the width direction Y, an interval D between a center of the detection electrode pair 42, 43 and a center of the detection electrode pair 44, 45 (refer to FIG. 5A described later) is set to, for example, 20 mm. The interval D corresponds to the interval between the first pulse wave sensor 40-1 and the second pulse wave sensor 40-2. Further, in the width direction Y, an interval between the detection electrode pair 42, 43 and an interval between the detection electrode pair 44, 45 are both set to, for example, 2 mm.

Such an electrode group 40E may be flatly configured and thus, in the blood pressure monitor 1, the belt 20 can be thinly configured as a whole. Further, the electrode group 40E may be flexibly configured, and thus the electrode group 40E does not impede compression of the wrist 90(left) by the compression cuff 21 nor impair the accuracy of the blood pressure measurement by the oscillometric method described later.

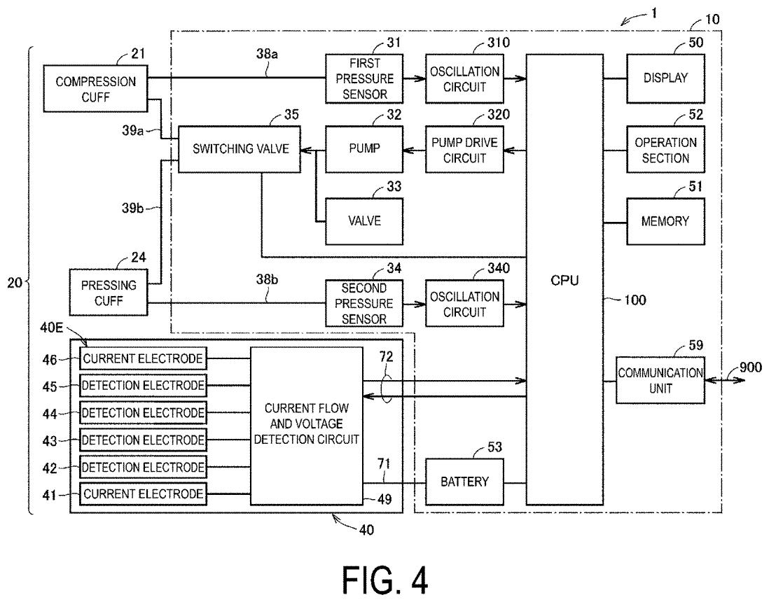

FIG. 4 is a diagram illustrating a block configuration of a control system of the blood pressure monitor 1 according to the first embodiment. The blood pressure monitor 1 includes an oscillometric method-based blood pressure measurement function and a PTT-based blood pressure measurement function. The blood pressure monitor 1 in FIG. 4 illustrates a configuration in which air is used as the fluid.

With reference to FIG. 4, the body 10 includes a central processing unit (CPU) 100 that functions as the control unit, the display 50, a memory 51 serving as a storage unit, the operating section 52, a battery 53, and a communication unit 59. Further, the body 10 includes a first pressure sensor 31, a pump 32, a valve 33, a second pressure sensor 34, and a switching valve 35. The switching valve 35 switches a connection destination of the pump 32 and the valve 33 to the compression cuff 21 or the pressing cuff 24.

Furthermore, the body 10 includes an oscillation circuit 310 and an oscillation circuit 340 that convert outputs from each of the first pressure sensor 31 and the second pressure sensor 34 into a frequency, and a pump drive circuit 320 that drives the pump 32. The sensor unit 40 includes the electrode group 40E and a current flow and voltage detection circuit 49.

The display 50 is configured by, for example, an organic electro-luminescence (EL) display and displays information in accordance with a control signal from the CPU 100. This information includes measurement results. Note that the display 50 is not limited to an organic EL display, and may be configured by other types of displays, such as a liquid crystal display (LCD), for example.

The operating section 52 is configured by, for example, a push type switch, and inputs an operation signal corresponding to an instruction for starting or stopping blood pressure measurement from the user to the CPU 100. Note that the operating section 52 is not limited to a push type switch, and may be, for example, a pressure sensitive type (resistance type) or a proximity type (capacitance type) touch panel switch, or the like. Further, the body 10 also includes a microphone (not illustrated) and may receive an instruction for starting blood pressure measurement by the voice of the user.

The memory 51 non-transitorily stores program data for controlling the blood pressure monitor 1, data used to control the blood pressure monitor 1, settings data for setting various functions of the blood pressure monitor 1, measurement result data of blood pressure values, and the like. Further, the memory 51 is used as a working memory or the like when the program is executed.

The CPU 100 executes various functions as a control unit in accordance with a program for controlling the blood pressure monitor 1 stored in the memory 51. For example, when executing blood pressure measurement by the oscillometric method, the CPU 100, upon receipt of an instruction for starting the blood pressure measurement from the operating section 52, drives the pump 32 (and the valve 33) on the basis of a signal from first pressure sensor 31. Further, the CPU 100 calculates the blood pressure values (maximum blood pressure (systolic blood pressure) and minimum blood pressure (diastolic blood pressure)) and calculates the pulse rate on the basis of the signals from the first pressure sensor 31.

When executing PTT-based blood pressure measurement, the CPU 100, in response to an instruction for starting blood pressure measurement from the operating section 52, executes control for driving the valve 33 to discharge air in the compression cuff 21. Further, the CPU 100 executes control for driving the switching valve 35 to switch the connection destination of the pump 32 (and valve 33) to the pressing cuff 24. Furthermore, the CPU 100 executes control for calculating the blood pressure value on the basis of a signal from the second pressure sensor 34.

The communication unit 59 is controlled by the CPU 100 to communicate with an external information processing device via a network 900. The external information processing device may include a portable terminal 10B and a server 30 described later, but the included devices are not limited thereto. Communication via the network 900 may include wireless or wired. For example, the network 900 may include the Internet and a local area network (LAN). Or the network 900 may also include one-to-one communication using a universal serial bus (USB) cable. The communication unit 59 may include a micro USB connector.

The pump 32 and the valve 33 are connected to the compression cuff 21 and the pressing cuff 24 via the switching valve 35 and air lines 39a, 39b. The first pressure sensor 31 and the second pressure sensor 34 are connected to the compression cuff 21 and the pressing cuff 24, respectively, via the air line 38a and the air line 38b, respectively. The first pressure sensor 31 detects the pressure in the compression cuff 21 via the air line 38a. The switching valve 35 is driven on the basis of a control signal imparted from the CPU 100, and switches the connection destination of the pump 32 and the valve 33 to the compression cuff 21 or the pressing cuff 24.

The pump 32 is comprised of a piezoelectric pump, for example. When the switching valve 35 switches the connection destination of the pump 32 and the valve 33 to the compression cuff 21, the pump 32 supplies air as a fluid for pressurization through the air line 39a to the compression cuff 21 to pressurize the pressure (cuff pressure) in the compression cuff 21. When the switching valve 35 switches the connection destination of the pump 32 and the valve 33 to the pressing cuff 24, the pump 32 supplies air through the air line 39b to the pressing cuff 24 to pressurize the pressure (cuff pressure) in the pressing cuff 24.

The valve 33 is mounted on the pump 32, and the opening and closing thereof is controlled in association with the pump 32 being turned on and off. Specifically, when the switching valve 35 switches the connection destination of the pump 32 and the valve 33 to the compression cuff 21, the valve 33 closes when the pump 32 is turned on, thereby filling air in the compression cuff 21, and opens when the pump 32 is turned off, thereby discharging air from the compression cuff 21 through the air line 39a and into the atmosphere.

When the switching valve 35 switches the connection destination of the pump 32 and the valve 33 to the pressing cuff 24, the valve 33 closes when the pump 32 is turned on, thereby filling air in the pressing cuff 24, and opens when the pump 32 is turned off, thereby discharging air from the pressing cuff 24 through the air line 39b and into the atmosphere. The valve 33 functions as a check valve, and the air discharged does not flow back. The pump drive circuit 320 drives the pump 32 on the basis of a control signal imparted from CPU 100.

The first pressure sensor 31 is, for example, a piezoresistive pressure sensor and is connected to the pump 32, the valve 33, and the compression cuff 21 via the air line 38a. The first pressure sensor 31 detects, via the air line 38a, a pressure of the belt 20 (compression cuff 21), such as a pressure with atmospheric pressure as reference (zero), for example, and outputs the pressure as a time series signal.

The oscillation circuit 310 outputs a frequency signal having a frequency corresponding to an electrical signal value based on a change in electrical resistance due to a piezoresistive effect from the first pressure sensor 31, to the CPU 100. The output of the first pressure sensor 31 is used to control the pressure of the compression cuff 21 and to calculate the blood pressure value by the oscillometric method.

The second pressure sensor 34 is, for example, a piezoresistive pressure sensor and is connected to the pump 32, the valve 33, and the pressing cuff 24 via the air line 38b. The second pressure sensor 34 detects, via the air line 38b, a pressure of the pressing cuff 24, such as a pressure with atmospheric pressure as reference (zero), for example, and outputs the pressure as a time series signal.

The oscillation circuit 340 oscillates in accordance with an electrical signal value based on a change in electrical resistance due to a piezoresistive effect from the second pressure sensor 34, and outputs a frequency signal having a frequency in accordance with an electrical signal value of the second pressure sensor 34, to the CPU 100. The output of the second pressure sensor 34 is used to control the pressure of the pressing cuff 24 and to calculate the blood pressure value based on PTT. When the pressure of the pressing cuff 24 is controlled for blood pressure measurement based on PTT, the CPU 100 controls the pump 32 and the valve 33 to pressurize and de-pressurize the cuff pressure according to various conditions.

The battery 53 supplies power to various elements mounted on the body 10. The battery 53 also supplies power to the current flow and voltage detection circuit 49 of the sensor unit 40 through a wire 71. The wire 71 is provided extending between the body 10 and the sensor unit 40 in the circumferential direction of the belt 20 in a state of being sandwiched between the band 23 of the belt 20 and the compression cuff 21, along with a wire 72 for signals.

### Summary of Blood Pressure Measurement Based on Pulse Transit Time

FIGS. 5A and 5B are schematic diagrams for explaining blood pressure measurement based on pulse transit time according to the first embodiment. Specifically, FIG. 5A is a schematic cross-sectional view in the longitudinal direction of the wrist when blood pressure measurement based on pulse transit time, is performed with the blood pressure monitor 1 attached to the wrist 90. FIG. 5B illustrates waveforms of pulse wave signals PS1, PS2. Note that in FIGS. 5A and 5B, the sensor unit 40 is positioned on the radial artery 91 of the measurement site.

With reference to FIG. 5A, the voltage detection circuit 49 applies a predetermined voltage to the current electrode pair 41, 46 using a booster circuit, a voltage adjustment circuit, or the like to flow a high frequency constant current i having, for example, a frequency of 50 kHz and a current value of 1 mA.

Further, the voltage detection circuit 49 detects a voltage signal v1 between the detection electrode pair 42, 43 constituting the first pulse wave sensor 40-1, and a voltage signal v2 between the detection electrode pair 44, 45 constituting the second pulse wave sensor 40-2. The voltage signals v1, v2 indicate a change in electrical impedance due to the pulse wave of the blood flow in the radial artery 91 at a portion of the palm side surface 90a of the wrist 90 (left) where the first pulse wave sensor 40-1 and the second pulse wave sensor 40-2 face each other.

Specifically, an amplifier 401 of the voltage detection circuit 49 is configured to include, for example, an op amp, and amplifies the voltage signals v1, v2. An analog filter 403 performs filtering processing on the voltage signals v1, v2 thus amplified. Specifically, the analog filter 403 removes noise other than the frequency characterizing the voltage signals v1, v2 (pulse wave signals) and performs the filtering processing to improve a signal-to-noise (S/N) ratio. An analog/digital (A/D) converter 405 converts the filtering-processed voltage signals v1, v2 from analog data to digital data, and outputs the digital data to the CPU 100 via the wire 72.

The CPU 100 carries out predetermined signal processing on the input voltage signals v1, v2 (digital data) to generate the pulse wave signals PS1, PS2 having mountain-shaped waveforms as illustrated in FIG. 5B.

Note that the voltage signals v1, v2 are about 1 my, for example. Further, each peak A1, A2 of the pulse wave signals PS1, PS2 is approximately 1 V, for example. Given that a pulse wave velocity (PWV) of the blood flow of the radial artery 91 is within a range of from 1000 cm/s to 2000 cm/s, the interval between the first pulse wave sensor 40-1 and the second pulse wave sensor 40-2 is D=20 mm and thus a time difference Δt between the pulse wave signal PS1 and the pulse wave signal PS2 is within a range of from 1.0 ms to 2.0 ms.

As illustrated in FIG. 5A, the pressing cuff 24 is in a pressurized state, and the compression cuff 21 is in a non-pressurized state with the air in an interior thereof discharged. The pressing cuff 24 and the solid material 22 are disposed across the first pulse wave sensor 40-1, the second pulse wave sensor 40-2, and the current electrode pair 41, 46 in an artery direction of the radial artery 91. Thus, when the pressing cuff 24 is pressurized by the pump 32, the first pulse wave sensor 40-1, the second pulse wave sensor 40-2, and the current electrode pair 41, 46 are pressed via the solid material 22 to the palm side surface 90a of the wrist 90.

The pressing force of each of the current electrode pair 41, 46, the first pulse wave sensor 40-1, and the second pulse wave sensor 40-2 on the palm side surface 90a of the wrist 90 can be set to an appropriate value. In the present embodiment, because the pressing cuff 24 of the fluid bag is used as the pressing portion, the pump 32 and the valve 33 can be commonly used with the compression cuff 21, and thus the configuration may be simplified. Further, because the first pulse wave sensor 40-1, the second pulse wave sensor 40-2, and the current electrode pair 41, 46 can be pressed via the solid material 22, the pressing force on the measurement site is uniform, and thus blood pressure measurement based on pulse transit time with high accuracy may be performed.

### Blood Pressure Measurement Operation Based on PTT

When the user provides instructions via the operating section 52 to perform blood pressure measurement based on PTT, the CPU 100 drives the switching valve 35 to switch the connection destination of the pump 32 and the valve 33 to the pressing cuff 24 in accordance with the instruction. Subsequently, the CPU 100 closes the valve 33, drives the pump 32 via the pump drive circuit 320, feeds air to the pressing cuff 24, and increases a cuff pressure Pc, which is the pressure in the pressing cuff 24, at a constant rate.

In this process of pressurization, the CPU 100 acquires the first and second pulse wave signals PS1, PS2 respectively output in time series by the first pulse wave sensor 40-1 and the second pulse wave sensor 40-2, and calculates the correlation coefficient r between the waveforms of the first and second pulse wave signals PS1, PS2 in real-time. When the CPU 100 determines that the correlation coefficient r calculated in real time in the process of pressurization exceeds a threshold Th (Th=0.99, for example), regarding the first and second pulse wave signals PS1, PS2 detected at the cuff pressure PC at that time, the time difference Δt between the peaks A1, A2 of the amplitudes of the first and second pulse wave signals PS1, PS2 is calculated as PTT (pulse transit time).

Further, the CPU 100 calculates (estimates) the blood pressure EBP based on PTT in accordance with a known equation (EBP=(α/(DT²)+β). In this equation, α and β are predetermined coefficients, and DT denotes the pulse transit time. As a result, blood pressure based on PTT is measured.

The CPU 100 repeatedly calculates the PTT and the blood pressure EBP as long as there is no instruction for stopping measurement via the operating section 52. The CPU 100 displays the blood pressure EBP on the display 50 and stores the blood pressure EBP in the memory 51. When an instruction for stopping measurement is input via the operating section 52, the CPU 100 controls each unit to stop the measurement operation.

Note that the sensor unit 40 utilizes an electrode for impedance measurement to measure the pulse wave signal, but is not limited thereto. For example, the sensor unit 40 may include a pressure sensor or an optical sensor to measure the pulse wave signal.

### Summary of Blood Pressure Measurement by Oscillometric Method

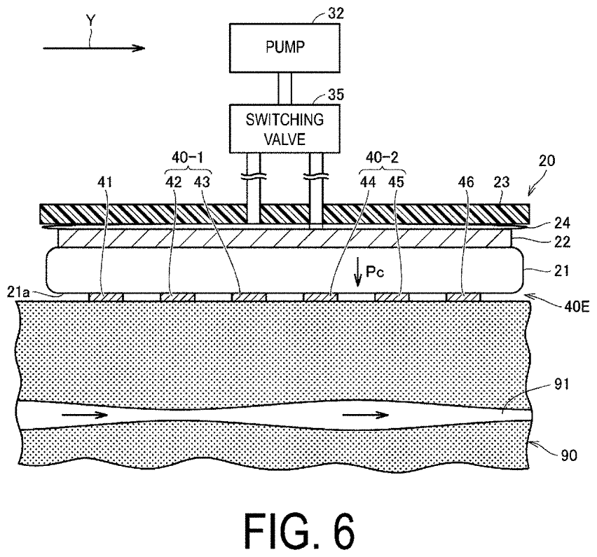

FIG. 6 is a schematic cross-sectional view of the blood pressure monitor 1 attached to the wrist 90, in a longitudinal direction of the wrist, when blood pressure is measured by the oscillometric method according to the first embodiment.

As illustrated in FIG. 6, the pressing cuff 24 is in a non-pressurized state with the air in the interior thereof discharged, and the pressing cuff 21 is in a pressurized state supplied with air. The compression cuff 21 extends circumferentially around the wrist 90 and, when pressurized by the pump 32, uniformly compresses the circumferential direction of the wrist 90 (left). With only the electrode group 40E between the inner peripheral surface of the compression cuff 21 and the wrist 90 (left), compression by the compression cuff 21 is not inhibited by other members and the blood vessels can be sufficiently closed.

In the blood pressure measurement by the oscillometric method, the CPU 100 calculates (estimates) the blood pressure according to the output waveform from the first pressure sensor 31 via the oscillation circuit 310 detected in the pressurization or de-pressurization process of the compression cuff 21 relative to the measurement site. The calculation method of the blood pressure by the oscillometric method according to the present embodiment is in accordance with a known method, and thus the description thereof is not repeated.

### Determination of Attachment State

In the first embodiment, the CPU 100 determines whether the blood pressure monitor 1 is in the attachment state. Specifically, when the belt 20 is wrapped around and attached to the measurement site, the belt 20 (compression cuff 21) is pressed against the measurement site. The first pressure sensor 31 detects this pressing force via the air line 38a.

The CPU 100 detects the pressing force from the output of the first pressure sensor 31 via the oscillation circuit 310. The CPU 100 compares the pressing force detected via the first pressure sensor 31 with a predetermined threshold P. When the results of the comparison satisfy the condition (Magnitude of pressing force>Threshold P), the CPU 100 determines that the blood pressure monitor 1 is in the attachment state and, when the results of the comparison satisfy the condition (Magnitude of pressing force≤Threshold P), the CPU 100 determines that the blood pressure monitor 1 is not in the attachment state. Thus, while the blood pressure monitor 1 is determined to be continually in the attachment state, the CPU 100 determines that the blood pressure monitor 1 is attached. The threshold P as used herein is acquired in advance by testing or the like.

While the method for determining the attachment state described above is a method of using the pressing force detected by the first pressure sensor 31, the method may be one of using the pressing force detected by the second pressure sensor 34. Alternatively, the method may be one of determining the attachment state on the basis of the pressing force detected by both the first pressure sensor 31 and the second pressure sensor 34.

Further, determination of the attachment state is not limited to determination on the basis of the magnitude of the pressing force described above. For example, the CPU 100 may determine whether the state is the attachment state (or attached) on the basis of a signal input by the user via the operating section 52.

### Determination of Attachment Position of Sensor Unit

As understood from the blood pressure measurement operation based on PTT described above, the accuracy of the measured blood pressure based on PTT depends on the detection accuracy of the waveform characteristics (correlation coefficient r and amplitude peak A1, A2) of the pulse wave signals output from the pulse wave sensors. Accordingly, accurate detection of the pulse wave signals is required. That is, it is necessary to dispose the first pulse wave sensor 40-1 and the second pulse wave sensor 40-2 of the sensor unit 40 over the measurement site (more particularly, the radial artery 91).

In view of the background described above, in the present embodiment, the CPU 100 displays guidance information for position adjustment on the display 50 for arranging the first pulse wave sensor 40-1 and the second pulse wave sensor 40-2 over the radial artery 91. The guidance information includes information for assisting with adjustment of the relative positional relationship between the sensor unit 40 and the measurement site so that the peak A1 of an amplitude of the first pulse wave signal PS1 in the first pulse wave sensor 40-1 and the peak A2 of an amplitude of the second pulse wave signal PS2 in the second pulse wave sensor are of a predetermined magnitude.

The user is assisted by the guidance information in adjusting the position of the sensor unit 40 in the attachment state. In this position adjustment, the user can move the position of the sensor unit 40 in an up-down direction (the direction in which the left arm extends) relative to the measurement site by pushing and shifting (sliding) a case of the display 50 in the attachment state in the width direction Y. Further, the user can move the position of the sensor unit 40 in a left-right direction (the direction substantially intersecting with the direction in which the left arm extends) relative to the measurement site by pushing and shifting (sliding) the case of the display 50 in the attachment state in the direction intersecting with the width direction Y.

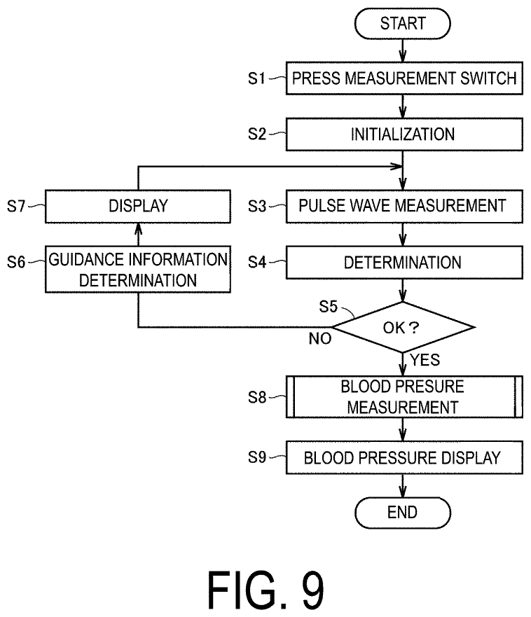

In the present embodiment, the guidance information includes information for evaluating the attachment state. Specifically, when the magnitudes of the amplitudes of the first pulse wave signal PS1 and the second pulse wave signal PS2 exceeds a threshold TA, the CPU 100 evaluates the attachment state as “OK”. On the other hand, when the magnitude of the amplitude of at least one of the first pulse wave signal PS1 and the second pulse wave signal PS2 is less than or equal to the threshold TA, the CPU 100 evaluates the attachment state as not good (“NG”). Note that the threshold TA is a value corresponding to a predetermined accuracy of the measured blood pressure based on PTT, and indicates a value obtained in testing. The user, by checking that the attachment state is “OK”, can identify that position adjustment was successful and, by checking that the attachment state is “NG”, can identify that position adjustment was not successful and continued adjustment is required. The CPU 100 does not activate the processing of blood pressure measurement while the attachment state is determined to be “NG”, and can activate the blood pressure measurement processing when the attachment state is determined to be “OK” (refer to step S8 in FIG. 9 described later). Accordingly, the evaluation results of the attachment state can be utilized in the determination of activation of blood pressure measurement processing.

The attachment state being evaluated as “OK” indicates that the relative positional relationship between the sensor unit 40 and the measurement site is a relationship in which the accuracy of blood pressure measurement based on PTT can be achieved. More specifically, the first pulse wave sensor 40-1 and the second pulse wave sensor 40-2 are positioned directly above the radial artery 91. On the other hand, the attachment state being evaluated as “NG” indicates that the relative positional relationship between the sensor unit 40 and the measurement site is a relationship in which the accuracy of blood pressure measurement based on PTT cannot be achieved. More specifically, both or one of the first pulse wave sensor 40-1 and the second pulse wave sensor 40-2 are not positioned directly above the radial artery 91. Note that, while evaluations of the attachment state are the two types of “OK” and “NG” here, there may be three or more types. Specifically, even in the case of “NG”, the magnitudes of the amplitudes of the first pulse wave signal PS1 and the second pulse wave signal PS2 may be classified as “NG-1”, “NG-2”, . . . on the basis of the magnitude of the difference from the threshold TA, starting with the smallest difference. Further, even in the case of “OK”, the magnitudes of the amplitudes of the first pulse wave signal PS1 and the second pulse wave signal PS2 may be classified on the basis of the magnitude of the difference from the threshold TA as “OK-1”, “OK-2”, . . . starting with the largest difference.

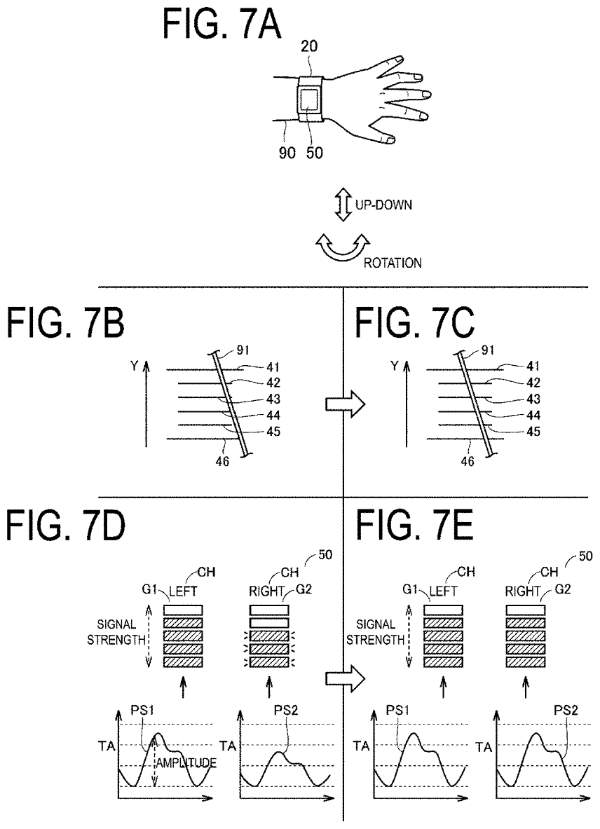

FIGS. 7A to 7E are diagrams for explaining the determination of the attachment state of the sensor unit 40 according to the first embodiment. As illustrated in FIG. 7A, in the first embodiment, the user visually checks, in the attachment state, the guidance information of the display 50 from above with the extending direction of the left arm parallel with a front surface of his or her body. In the state of FIG. 7A, according to the spaced-apart arrangement at the interval D described above, the first pulse wave sensor 40-1 is positioned on the left side of the user and the second pulse wave sensor 40-2 is likewise positioned on the right side. In this context, the CPU 100 displays first indicator information G1 indicating the magnitude of the amplitude of the first pulse wave signal PS1 on the left side of the user on the screen of the display 50, and second indicator information G2 indicating the magnitude of the amplitude of the second pulse wave signal PS2 on the right side of the user on the screen of the display 50 (refer to FIG. 7D and FIG. 7E described later).

In this manner, the CPU 100 displays the first and second indicator information G1, G2 in positions aligned with the spaced apart arrangement described above of the first pulse wave sensor 40-1 and the second pulse wave sensor 40-2, in accordance with the direction in which the user visually checks the information of the display 50 in FIG. 7A. In other words, “displays in positions aligned” is equivalent to respectively displaying the first indicator information G1 and the second indicator information G2 on the screen of the display 50 in positions respectively corresponding to the first pulse wave sensor 40-1 and the second pulse wave sensor 40-2 spaced apart at the interval D.

In the attachment state, as illustrated in FIG. 7B, when the first pulse wave sensor 40-1 is positioned directly above the radial artery 91 and the second pulse wave sensor 40-2 is not positioned directly above the radial artery 91, the amplitude of the first pulse wave signal PS1 indicates a magnitude exceeding the threshold TA, but the magnitude of the amplitude of the second pulse wave signal PS2 does not exceed the threshold TA, as illustrated in the lower area of FIG. 7D. Accordingly, the CPU 100 evaluates the attachment state in FIG. 7B as “NG”.

When the attachment state of FIG. 7B is evaluated as “NG”, the CPU 100 displays the first indicator information G1 and the second indicator information G2 respectively corresponding to the first pulse wave signal PS1 and the second pulse wave signal PS2 in the display 50 by pictogram groups, as illustrated in the upper area of FIG. 7D. The pictogram group is a row consisting of a plurality of pictograms having a rectangular shape, and the CPU 100 displays the pictogram group extending in a direction intersecting with the Y direction. The CPU 100 detects the magnitude of the amplitude of each of the first pulse wave signal PS1 and the second pulse wave signal PS2 and illuminates one or more pictograms in the corresponding pictogram group according to the magnitude of the amplitude of the pulse wave signal. Thus, the magnitude of the amplitude of the corresponding pulse wave signal is indicated by the number of pictograms illuminated in the row of the pictogram group.

Preferably, illumination modes of the pictogram group differ for a pictogram indicating that the amplitude exceeds the threshold TA and a pictogram indicating that the amplitude is the threshold TA or less. In FIGS. 7A to 7E, for example, a pictogram indicating that the amplitude exceeds the threshold TA stays illuminated, and a pictogram indicating that the amplitude is the threshold TA or less blinks. In addition to staying illuminated and blinking, display modes may include a change in display color. Note that the shape of the pictogram is not limited to a rectangle.

Further, the CPU 100 displays a character CH indicating, as the position of the corresponding pulse wave sensor, the “left” or “right” side of the user in association with the first indicator information G1 and the second indicator information G2 in each pictogram group. In the upper area of FIG. 7D, the CPU 100 can guide the user by, for example, indicating that the pulse wave amplitude values detected by the first pulse wave sensor 40-1 and the second pulse wave sensor 40-2 are not balanced, or that the pulse wave amplitude values are relatively high or low, and thus motivate the user to move the position of the sensor unit 40. The motivated user pushes the case of the display 50 rightward to increase the amplitude of the pulse wave signal indicated by the indicator information G2 with which the character CH indicating “right” is associated.

When the display 50 is pushed rightward, the position of the sensor unit 40 moves to the left of the user. In conjunction with this movement, the position of the sensor unit 40 (the first pulse wave sensor 40-1 and the second pulse wave sensors 40-2) is moved directly above the radial artery 91, as illustrated in FIG. 7B and FIG. 7C.

When the sensor unit 40 is positioned directly above the radial artery 91, the amplitudes of the first pulse wave signal PS1 and the second pulse wave signal PS2 become sufficiently large, and the first indicator information G1 and the second indicator information G2 exceed the threshold TA illustrated in the lower area of FIG. 7E. At this time, as illustrated in FIG. 7E, the first indicator information G1 and the second indicator information G2 indicate that the amplitudes of the first pulse wave signal PS1 and the second pulse wave signal PS2 are sufficiently large. At this time, the CPU 100 determines that the attachment state in FIG. 7C is “OK”. The user checks the first indicator information G1 and the second indicator information G2 in the upper half of FIG. 7E and thus receives guidance that the attachment state is good.

The CPU 100 performs blood pressure measurements (estimates) based on PTT in accordance with the characteristics of the waveforms of the first pulse wave signal PS1 and the second pulse wave signal PS2 detected when the attachment state is determined to be “OK” and the known equation described above.

In this manner, the user is motivated to adjust the relative positional relationship between the sensor unit 40 and the measurement site from the first indicator information G1 and the second indicator information G2, and receives adjustment guidance from the first indicator information G1, the second indicator information G2, and the characters CH. Further, because the characters CH are displayed on the same screen in association with the indicator information, the user can check the guidance information for positional adjustment without switching screens.

### Functional Configuration of CPU 100

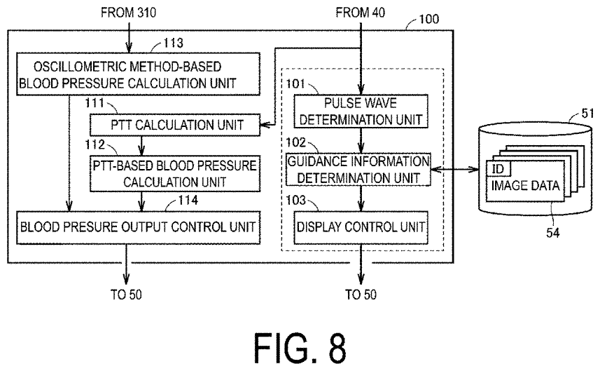

FIG. 8 is a diagram schematically illustrating a configuration of a function for outputting the guidance information in association with the blood pressure measurement function according to the first embodiment. With reference to FIG. 8, the CPU 100 includes, as a function for outputting the guidance information, a pulse wave determination unit 101, a guidance information determination unit 102 configured to determine the guidance information using image data 54 of the memory 51, and a display control unit 103. Further, the CPU 100 includes, as a blood pressure measurement function, a PTT calculation unit 111 that calculates PTT according to the processing described above, a PTT-based blood pressure calculation unit 112 that calculates (estimates) blood pressure on the basis of PTT according to the known equation described above, an oscillometric method-based blood pressure calculation unit 113 that calculates (estimates) blood pressure on the basis of the oscillometric method described above, and a blood pressure output control unit 114.

The memory 51 stores the image data 54 corresponding to each (Magnitude of the amplitude of the first pulse wave signal PS1, Magnitude of the amplitude of the second pulse wave signal PS2) set that can be detected. The set corresponding to the image data 54 is indicated by an identification (ID) assigned to the image data 54. The image data 54 includes the pictogram groups indicating the magnitudes of the amplitudes of the first pulse wave signal PS1 and the second pulse wave signal PS2, and images of the characters CH indicating “left” and “right”. The image data 54 corresponding to each (Magnitude of the amplitude of the first pulse wave signal PS1, Magnitude of the amplitude of the second pulse wave signal PS2) set is generated on the basis of testing and the like, and stored in association with the set in the memory 51.

The pulse wave determination unit 101 compares the magnitudes of the amplitudes of the first pulse wave signal PS1 and the second pulse wave signal PS2 with the threshold TA and determines whether a condition (Magnitude of amplitude>Threshold TA) is satisfied on the basis of the comparison result. When the magnitudes of the amplitudes of the first pulse wave signal PS1 and the second pulse wave signal PS2 satisfy the condition, the attachment state is the preferred attachment state for blood pressure measurement based on PTT, that is, an attachment state that can maintain measurement accuracy.

When it is determined that the condition described above is not satisfied by the pulse wave determination unit 101, the guidance information determination unit 102 determines the guidance information to be outputted. Specifically, the guidance information determination unit 102 detects the amplitudes of the first pulse wave signal PS1 and the second pulse wave signal PS2 and searches the memory 51 on the basis of the detected (Magnitude of the amplitude of the first pulse wave signal PS1, Magnitude of the amplitude of the second pulse wave signal PS2) combination. The guidance information determination unit 102 reads the image data 54 to which the ID matching the combination is assigned from the memory 51.

On the basis of the image data 54 from the guidance information determination unit 102, the display control unit 103 generates a control signal for causing the display 50 to perform display. With the display 50 driven in accordance with the control signal, the first indicator information G1 and the second indicator information G2 based on the detected magnitudes of the amplitudes of the first pulse wave signal PS1 and the second pulse wave signal PS2 are displayed on the display 50.

The functions of each component in FIG. 8 are stored as programs in the memory 51. The CPU 100 reads out the programs from the memory 51, thereby realizing the function of each component. Note that the functions of each component are not limited to a method of being realized by a program. For example, the functions may be realized by a circuit including an application specific integrated circuit (ASIC) or a field-programmable gate array (FPGA). Furthermore, the functions may be realized by a combination of a program and a circuit.

Note that the guidance information is not limited to the image data 54 stored in the memory 51. For example, an image generation program including a script program may be executed to generate image data for display control. In this case, the (Magnitude of the amplitude of the first pulse wave signal PS1, Magnitude of the amplitude of the second pulse wave signal PS2) combination described above is a parameter (argument or the like) of the script program.

### Processing Flowchart

FIG. 9 is a flowchart illustrating the processing of blood pressure measurement based on the output of the guidance information and PTT according to the first embodiment. The program according to this flowchart is stored in the memory 51 and is read and executed by the CPU 100.

With reference to FIG. 9, first the CPU 100 receives a start instruction when the user uses the operating section 52 to perform a switch operation for starting blood pressure measurement by PTT in the attachment state (step S1). The CPU 100 performs the initialization processing when blood pressure measurement is started (step S2). For example, air is discharged from the cuff.

The CPU 100 starts the processing of pulse wave measurement for PTT (step S3). The pulse wave determination unit 101 acquires the first pulse wave signal PS1 and the second pulse wave signal PS2 thus measured from the sensor unit 40, and determines whether the (Amplitude>Threshold TA) condition of the magnitudes of the amplitudes of the first pulse wave signal PS1 and the second pulse wave signal PS2 thus acquired is satisfied (step S4).

On the basis of the output of the pulse wave determination unit 101, the CPU 100 determines whether the condition described above is satisfied (step S5). When it is determined that the condition is satisfied (YES in step S5), the CPU 100 evaluates the attachment state as “OK” and performs blood pressure measurement based on PTT in step S8 described later.

On the other hand, when it is determined that the condition is not satisfied (NO in step S5), the CPU 100 evaluates the attachment state as “NG”. Further, the guidance information determination unit 102 determines the guidance information (step S6), and the display control unit 103 controls the display of the display 50 in accordance with the guidance information (step S7). Subsequently, the processing proceeds to step S3 in which the subsequent processing is performed in the same way as described above.

When blood pressure measurement based on PTT is performed (step S8), the measured blood pressure is displayed on the display 50 (step S9). Further, the measurement result is stored in the memory 51. In step S8, PTT is calculated by the PTT calculation unit 111, and the blood pressure based on the calculated PTT is calculated (estimated) by the PTT-based blood pressure calculation unit 112.

Note that while the PTT-based blood pressure measurement (step S8) is not performed during the period in which the attachment state is evaluated as “NG” in FIG. 9, calculation of the PTT by the PTT calculation unit 111 and calculation (estimation) of the blood pressure by the PTT-based blood pressure calculating unit 112 based on the PTT may be performed even when the attachment state is evaluated as “NG”. For example, when the attachment state is evaluated as “NG” a predetermined number of consecutive times or when the period over which the attachment state has been evaluated as “NG” exceeds a predetermined period (for example, when a predetermined period after reception of the start instruction (step S1) has been exceeded), PTT-based blood pressure measurement (step S8) may be performed with the attachment state evaluated as “NG”.

### Display Example of Guidance Information

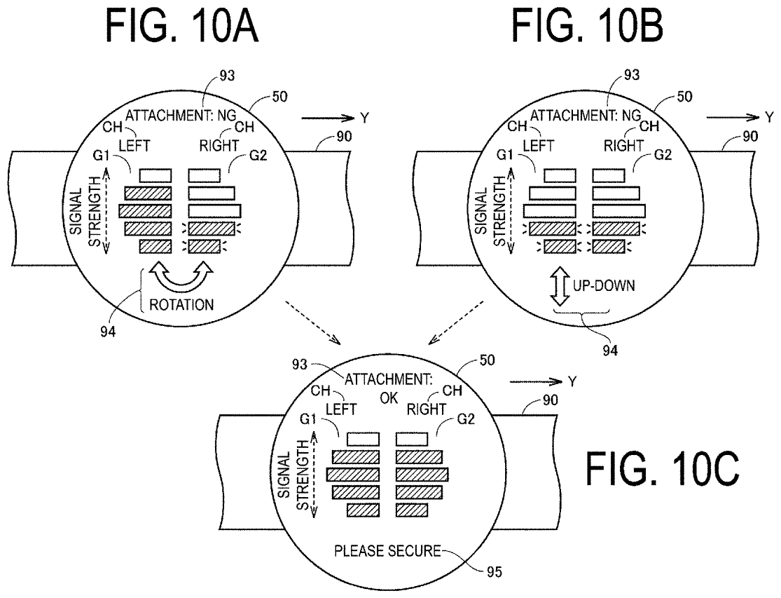

FIGS. 10A to 10C, FIGS. 11A to 11C, and FIGS. 12A to 12C are diagrams illustrating another display example of guidance information according to the first embodiment. FIGS. 10A to 10C illustrate a case in which the case (screen) of the display 50 is circular. FIG. 10A and FIG. 10B are display examples of a case in which the attachment state is “NG”, and information 93 indicating the evaluation (“NG”) of the attachment state and information 94 providing guidance with regard to the direction in which the position of the sensor unit 40 is to be moved relative to the measurement site are displayed on the same screen of the display 50 with other information (the indicator information G1, G2 and the characters CH). The information 94 in FIG. 10A is information that provides guidance for rotating the circular case of the display 50 concentrically around a center of the circle, and is indicated by an arrow mark indicating the rotation direction. In contrast, the information 94 in FIG. 10B is information that provides guidance for moving the case of the display 50 in the up-down direction, and is indicated by an arrow mark indicating the up-down direction.

FIG. 10C is a display example of a case in which the attachment state is evaluated as “OK”, and information 95 providing guidance for securing the position of the sensor unit 40 is displayed on the same screen of the display 50 with other information (the indicator information G1, G2 and the characters CH) instead of the information 93 indicating the evaluation (“OK”) of the attachment state and the information 94 of the direction of movement described above. The information 95 providing guidance for securing is, for example, “Please secure”, but is not limited thereto.

Note that when the attachment state is evaluated as “OK”, the information 95 providing guidance for securing the position of the sensor unit 40 is displayed on the display 50 instead of the information 93 and the information 94 displayed when the attachment state is evaluated as “NG”.

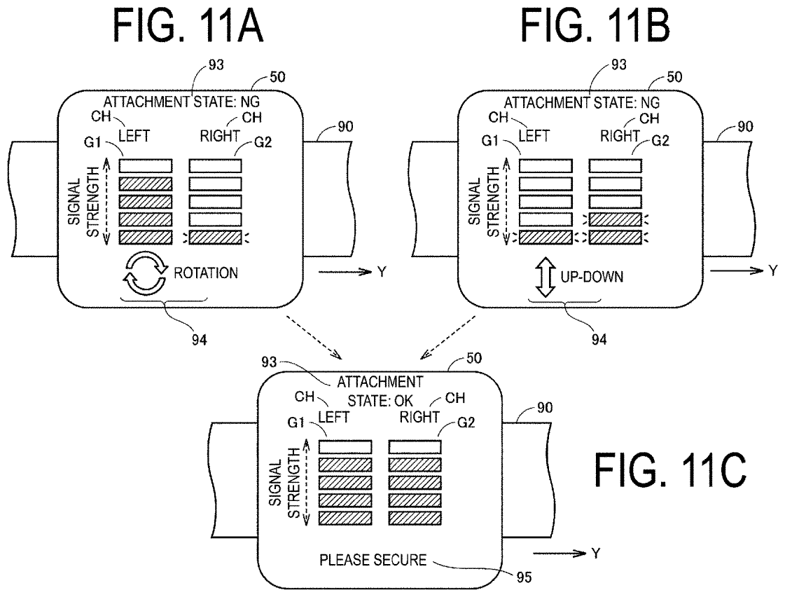

FIGS. 11A to 11C illustrate a case in which the case (screen) of the display 50 is rectangular. FIG. 11A and FIG. 11B are display examples of when the attachment state is evaluated as “NG”. The information 94 in FIG. 11A is information that provides guidance for rotating the rectangular case of the display 50 concentrically around a center of the rectangle, and is indicated by arrow marks (a set of two arrow marks differing in orientation) indicating the rotation direction. FIG. 11C is a display example of when the attachment state is evaluated as “OK”.

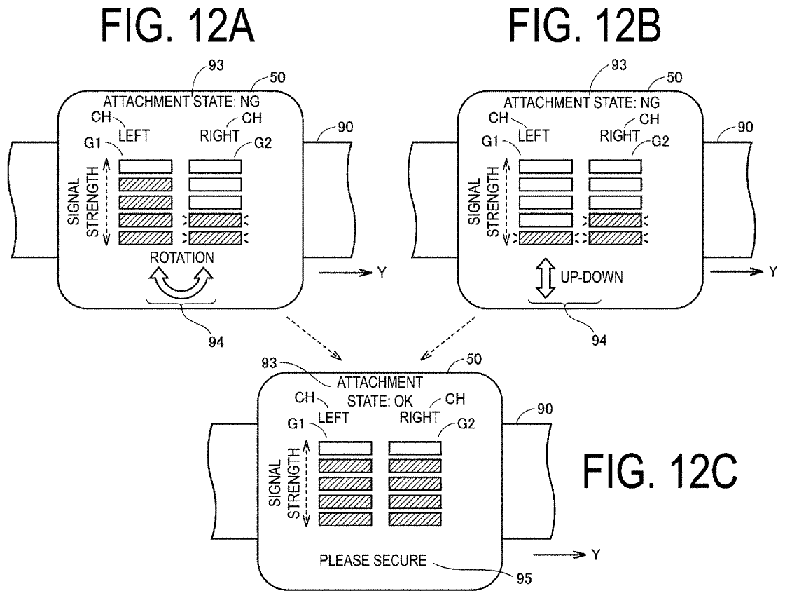

FIGS. 12A to 12C are modified examples of FIGS. 11A to 11C. In FIG. 12A, the information 94 is indicated by one arrow indicating both directions, rather than the set of two arrow marks of the information 94 in FIG. 11A having different orientations.

In this way, when the information 93 provides guidance with regard to the need for the position of the sensor unit 40 to be adjusted relative to the measurement site, the user is guided by the information 94 on the same screen as to whether the movement should be performed in the up-down direction or rotationally. Thus, the user may manipulate the case of the display 50 in accordance with the direction of movement of the information 94 to efficiently adjust the relative positional relationship between the sensor unit 40 and the measurement site so that the attachment state is “OK”. Note that the guidance information (corresponding to the icon of the arrow mark) related to whether movement is to be performed in the up-down direction or rotationally is also information determined on the basis of the (Magnitude of the amplitude of the first pulse wave signal PS1, Magnitude of the amplitude of the second pulse wave signal PS2) combination, and is included in advance in the image data 54 described above in association with the set.

While the display described above is when the blood pressure monitor 1 is attached to the wrist 90 (left), the same can be implemented when attached to the right wrist. In that case, the arrangement mode of the first pulse wave sensor 40-1 and the second pulse wave sensor 40-2 relative to the measurement site is opposite to that when attached to the wrist 90 (left), and thus the display positions on the display 50 of the first indicator information G1 and the second indicator information G2 corresponding to the output of each pulse wave sensor are reversed. Further, the CPU 100 can determine, by user input from the operating section 52, whether the site where the blood pressure monitor 1 is attached is the left wrist or the right wrist. Alternatively, the blood pressure monitor 1 may be equipped with an acceleration sensor to allow the CPU 100 to determine whether attachment is on the left side or the right side from an output of the acceleration sensor.

### Storage Example of Measurement Result

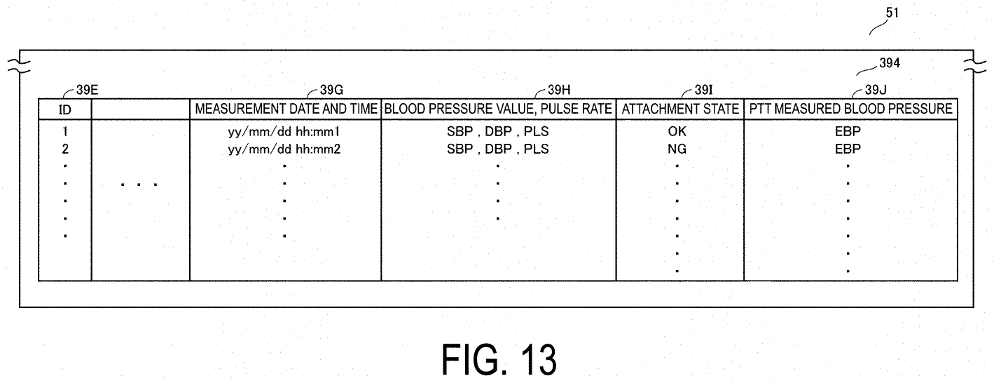

FIG. 13 is a diagram illustrating a storage example of a measurement result according to the first embodiment. With reference to FIG. 13, the memory 51 stores a table 394 that records the measurement results of the blood pressure monitor 1. With reference to FIG. 13, the table 394 stores the measurement data in units of records. Each record includes data 39E of an identification (ID) for uniquely identifying the record, data 39G of the measurement date and time, data 39H including a blood pressure value (systolic blood pressure SBP and diastolic blood pressure DBP) calculated (estimated) by the oscillometric method-based blood pressure calculation unit 113, data 39I indicating “OK” or “NG” serving as an evaluation of the attachment state during PTT-based blood pressure measurement, and data 39J indicating the blood pressure value calculated (estimated) by the PTT-based blood pressure calculation unit 112.

The blood pressure output control unit 114 stores, in association with the data 39G of the measurement date and time, the data 39H of the blood pressure and the pulse rate according to the oscillometric method, measured at the date and time, as well as the data 39J of the PTT-based blood pressure value, in the memory 51.

The mode of storing the measurement data in the table 394 is not limited to units of records such as illustrated in FIG. 13. Each time the blood pressure is measured, the detected data 39E to 39J may be associated (linked).

### Other Display Examples

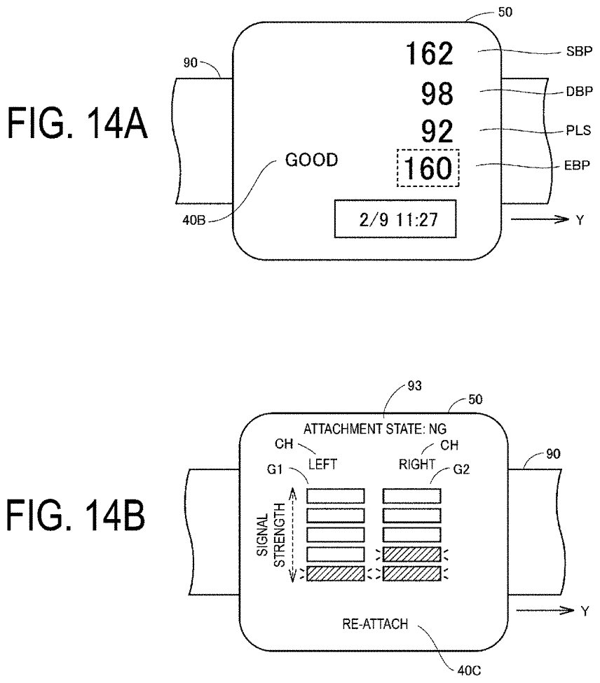

FIGS. 14A and 14B are diagrams illustrating another display example according to the first embodiment. With reference to FIG. 14A, on the screen of the display 50, an evaluation 40B of the attachment state, the systolic blood pressure SBP, the diastolic blood pressure DBP, the pulse rate PLS, the PTT-based measured blood pressure EBP, and the measurement date and time data are displayed as a measurement result. In FIG. 14A, according to the evaluation 40B of the attachment state, the “OK” evaluation of the attachment state is indicated by the characters “GOOD”. From the information of the evaluation 40B of the attachment state, the user can also obtain a measure of confidence regarding whether the displayed blood pressure EBP is a reliable value.

The display example in FIG. 14A corresponds to, for example, a display example when the blood pressure measurement has ended (step S9) or a display example of data read from the table 394 in FIG. 13. The blood pressure information in FIG. 14A is displayed by the blood pressure output control unit 114 controlling the display 50. Specifically, the blood pressure output control unit 114 generates display data based on the blood pressure calculated by the PTT-based blood pressure calculation unit 112 or the oscillometric method-based blood pressure calculation unit 113, and drives the display 50 on the basis of the display data. Alternatively, the blood pressure output control unit 114 generates display data based on the data 39H and the data 39J associated in the table 394 in FIG. 13, and drives the display 50 on the basis of the generated display data. Thus, the blood pressure output control unit 114 can display the measured blood pressure data or the blood pressure data stored in the table 394 on the display 50.

### Yet Another Display Example

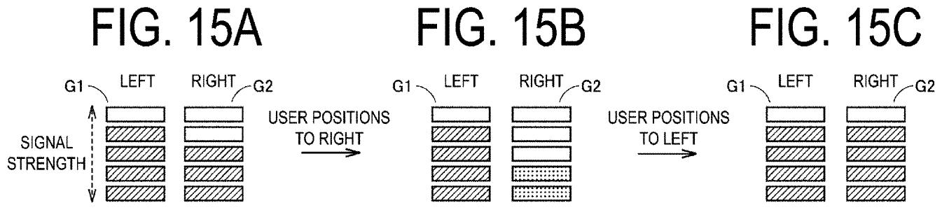

FIGS. 15A to 15C are diagrams illustrating yet another display example according to the first embodiment. The guidance information in FIGS. 15A to 15C not only presents the amplitude value (signal strength) of the first pulse wave signal PS1 or the second pulse wave signal PS2 from the first pulse wave sensor 40-1 or the second pulse wave sensor 40-2, but also changes in the amplitude value.

Specifically, FIGS. 15A to 15C illustrate a case in which guidance information based on amplitude values changes from FIG. 15A to FIG. 15B to FIG. 15C, in accordance with position adjustments of the sensor unit 40 performed by the user in accordance with the guidance information. Specifically, in FIG. 15A, for example, the amplitude value (signal strength) of the first pulse wave signal PS1 exceeds the threshold TA (here, equivalent to four pictograms), but the amplitude value of the second pulse wave signal PS2 is less than the threshold TA.

The user adjusts the position of the sensor unit 40 by pushing the display 50 rightward in accordance with the guidance information in FIG. 15A. As a result, when the CPU 100 determines that the amplitude value of the second pulse wave signal PS2 has changed and decreased from the amplitude value before the movement illustrated in FIG. 15A, the CPU 100 changes the color of the pictogram of the second indicator information G2 as in FIG. 15B. This change in display color can provide guidance to the user that the position adjustment is not appropriate.

The user adjusts the position of the sensor unit 40 by pushing the display 50 leftward in accordance with the change in color of the second indicator information G2 in FIG. 15B. As a result, the position of the sensor unit 40 (the first pulse wave sensor 40-1 and the second pulse wave sensors 40-2) is moved directly above the radial artery 91. In this case, when the CPU 100 determines that the amplitude value of the second pulse wave signal PS2 has changed and increased from the amplitude value before the movement illustrated in FIG. 15B, the CPU 100 changes the pictogram of the second indicator information G2 to the original color as in FIG. 15C. This guidance information in FIG. 15C can provide guidance to the user that the position adjustment is appropriate.

### Yet Another Display Example

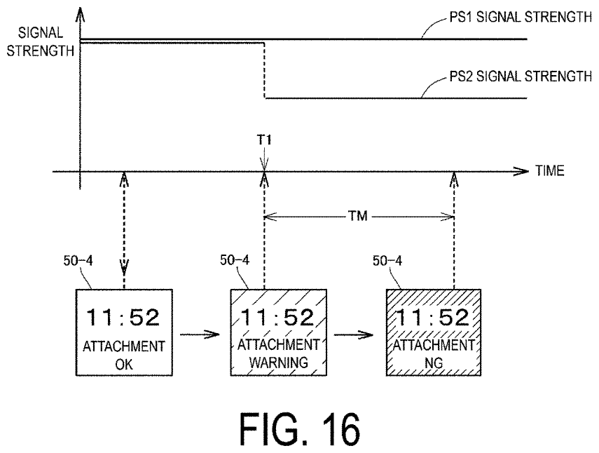

FIG. 16 is a diagram illustrating yet another display example according to the first embodiment. In FIG. 16, changes in the signal strengths (pulse wave amplitude values) of the first pulse wave signal PS1 and the second pulse wave signal PS2, and changes in the display mode of an icon 50-4 indicating an evaluation of the attachment state in association therewith are displayed.

Specifically, the signal strengths (pulse wave amplitude values) of the first pulse wave signal PS1 and the second pulse wave signal PS2 exceed the threshold TA from the start of attachment to a time T1. Thus, from the start of attachment to the time T1, the CPU 100 displays the icon 50-4 of the display 50 using the characters and color indicating the attachment state “OK”. When the pulse wave amplitude value of the second pulse wave signal PS2 at the time T1 becomes less than or equal to the threshold TA, the CPU 100 changes the icon 50-4 of the display 50 to the characters and color indicating the attachment state “Caution”. Further, when the CPU 100 determines that a state in which the pulse wave amplitude value is less than or equal to the threshold TA continues for a predetermined period (for example, a period TM) from the time T1, the CPU 100 changes the icon 50-4 of the display 50 to the characters and color indicating the attachment state “NG”.

According to FIGS. 15A to 16, while the guidance information is displayed, upon changing the magnitude of the amplitude of the pulse wave signal of the first pulse wave signal PS1 or the magnitude of the amplitude of the pulse wave signal of the second pulse wave signal PS2, the guidance information can also include information providing notification of the change. Thus, guidance to the user in relation to a change in evaluation of the attachment state may be provided by a change in characters and color by the icon 50-4.

Note that in a case where guidance is provided by vibration of the device (the blood pressure monitor 1 or the portable terminal 10B described later), a strength or cycle of the vibration may be changed in conjunction with the change in the guidance information by the icon 50-4.

### Yet Another Display Example

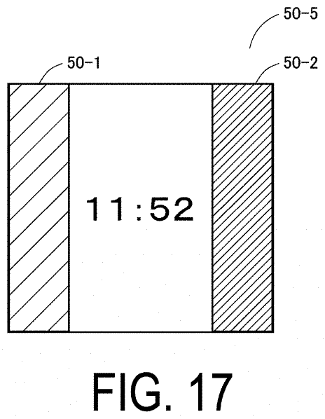

In the first embodiment, information for evaluating the attachment state is displayed in association with information for evaluating the blood pressure EBP measured on the basis of PTT. FIG. 17 is a diagram illustrating yet another display example according to the first embodiment. In FIG. 17, an icon 50-5 displayed on the display 50 includes a portion 50-1 indicating an evaluation of the attachment state and a portion 50-2 indicating an evaluation of the blood pressure EBP measured on the basis of PTT. The CPU 100 changes the color of the portion 50-1 of the evaluation of the attachment state of the icon 50-5 and the color of the portion 50-2 of the evaluation of the blood pressure EBP according to the contents of the corresponding evaluations. Guidance to the user related to the evaluation of the attachment state (“OK”, “NG”, “Re-attach”, and the like.) while associating the evaluation (normal blood pressure, high blood pressure, or the like) of the measured blood pressure EBP may be provided.

### Modified Example of Determination Period of Attachment State

The period for determining the attachment state of the blood pressure monitor 1 is not limited to during blood pressure measurement based on PTT described above (refer to FIG. 9). For example, during the period that it is determined that the blood pressure monitor 1 is attached to the user, the CPU 100 may periodically determine the attachment state and display the guidance information described above on the basis of the determination results.

More specifically, while the blood pressure monitor 1 is determined to be attached, the pulse wave determination unit 101 of the CPU 100 continually (for example, at regular constant intervals) determines whether the (Magnitude of amplitude>Threshold TA) condition is satisfied. Then, the guidance information determination unit 102 sets the guidance information including the evaluation (OK or NG) of the attachment state, and the display control unit 103 displays the guidance information on the display 50. This display mode is, for example, illustrated in FIGS. 10A and 10B, FIGS. 11A and 11B, or FIG. 12A and FIG. 12B.

Further, when the peak A1 of the pulse wave signal PS1 or the peak A2 of the pulse wave signal PS2 detected in the attachment state of the blood pressure monitor 1 is determined to be less than the threshold TA, or less than the threshold TA a predetermined number of times in a row, the CPU 100 displays an alarm 40C (or error) prompting the wrapping and attaching on the display 50 (refer to FIG. 14B) again.

Alternatively, when the peak A1 of the pulse wave signal PS1 or the peak A2 of the pulse wave signal PS2 is less than the threshold TA1 (<TA), or when the peak A1 of the pulse wave signal PS1 or the peak A2 of the pulse wave signal PS2 is less than the threshold TA1 (<TA) a predetermined number of times in a row, the CPU 100 displays the alarm 40C (or error) on the display 50 (refer to FIG. 14B).

While, in FIG. 14B, the character message “Re-attach” is indicated as the alarm 40C, the character message is not limited thereto and may be other information including a predetermined image (video, still image).

While the blood pressure monitor 1 is attached, the user can always check the appropriateness of the attachment state or the need for re-attachment by checking the screen of the display 50. Note that the output mode of the information 93 or the alarm 40C indicating the evaluation of the attachment state (“OK” or “NG”) while the blood pressure monitor 1 is attached is not limited to the display of the display 50, and may be another mode including audio output or vibration.

### Second Embodiment

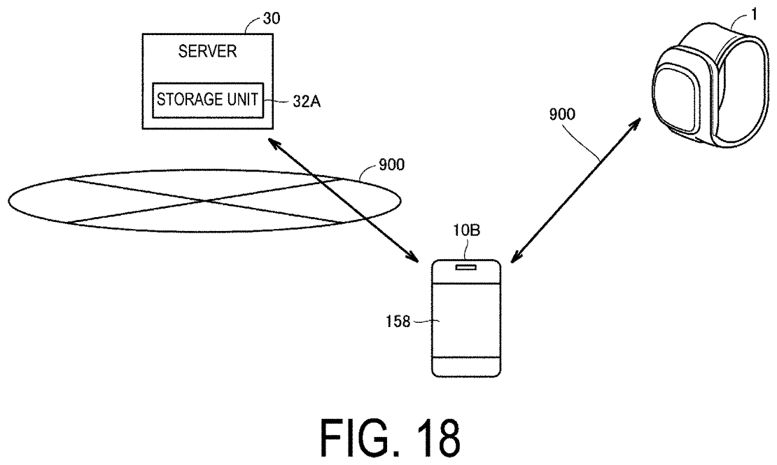

FIG. 18 is a diagram illustrating a schematic configuration of a system according to a second embodiment. The blood pressure monitor 1 described above communicates via the network 900 with the portable terminal 10B or the server 30, which is an external information processing device. In the system of FIG. 18, the blood pressure monitor 1 communicates with the portable terminal 10B via the LAN, and the portable terminal 10B communicates with the server 30 via the Internet. Thus, the blood pressure monitor 1 can communicate with the server 30 via the portable terminal 10B. Note that the blood pressure monitor 1 may communicate with the server 30 not via the portable terminal 10B.

While, in the first embodiment described above, the information 93 of “OK” or “NG” or the alarm 40C indicating the evaluation of the attachment state while the blood pressure monitor 1 is attached is displayed on the display 50 of the blood pressure monitor 1, the CPU 100 may transmit the information 93 or the alarm 40C of the evaluation of the attachment state to the portable terminal 10B and cause a displaying section 158 to display the information 93 or the alarm 40C. As a result, the blood pressure monitor 1 can output an evaluation of the attachment state from the display of the displaying section 158 of the portable terminal 10B. Further, the portable terminal 10B may provide notification of the information 93 or the alarm 40C of the evaluation of the attachment state using other output modes including vibration or audio of the portable terminal 10B.

While, in the first embodiment described above, the measurement result (FIG. 14A) is displayed on the display 50 of the blood pressure monitor 1, the display destination may be the displaying section 158 of the portable terminal 10B, and may be both the display 50 and the displaying section 158. Further, the storage destination of the measurement result shown in table 394 in FIG. 13 is not limited to the memory 51 of the blood pressure monitor 1. For example, the storage destination may be the storage unit of the portable terminal 10B or a storage unit 32A of the server 30. Or, the result may be stored in two or more of the memory 51, the storage unit of the portable terminal 10B and the storage unit 32A of the server 30.

### Third Embodiment

In the embodiments described above, a program may be provided that causes a computer to function and execute controls such as those described in the flowcharts described above. Such a program can also be provided as a program product stored on a non-transitory computer-readable recording medium, such as a compact disk read only memory (CD), a secondary storage device, a main storage device, a memory card, and the like attached to a computer of the blood pressure monitor 1. Alternatively, a program may be provided, which is stored on a recording medium such as a hard disk built into a computer. Further, the program may also be provided by download via the network 900.

The embodiments described herein are illustrative in all respects and are not intended as limitations. The scope of the present disclosure is indicated not by the descriptions above but by the claims and includes all meaning equivalent to the scope and changes within the scope.

### REFERENCE SIGNS LIST

1 Blood pressure monitor
10B Portable terminal
20 Belt
20a, 23a, 24a Inner peripheral surface
20b, 21a Outer peripheral surface
21 Compression cuff
24 Pressing cuff
30 Server
40-1 First pulse wave sensor
40-2 Second pulse wave sensor
40 Sensor unit
40B Evaluation of attachment state
50 Display
51 Memory
52 Operating Section
54 Image data
59 Communication unit
40C Alarm
90 Wrist
91 Radial artery
101 Pulse wave determination unit
102 Guidance information determination unit
103 Display control unit
158 displaying section
900 Network
A1, A2 Peak
CH Character
D Interval
DBP Diastolic blood pressure
EBP Blood pressure
G1 First indicator information
G2 Second indicator information
PLS Pulse rate
PS1 First pulse wave signal
PS2 Second pulse wave signal
SBP Systolic blood pressure
TA, Th Threshold
i Constant current
r Correlation coefficient
v1, v2 Voltage signal

1. A display control device provided to a measurement device, the measurement device comprising:
a belt wrapped around and attached to a measurement site for measuring a pulse transit time;
a sensor unit provided on an inner peripheral surface of the belt that is toward the measurement site when the belt is attached; and
a display provided on an outer peripheral surface of the belt opposite to the inner peripheral surface,
the display provided on the outer peripheral surface in a portion configured to face a portion where the sensor unit being positioned when the belt is attached, and
the sensor unit including a first pulse wave sensor and a second pulse wave sensor disposed spaced apart from each other in a width direction of the belt, wherein
the display control device configured to display first indicator information indicating a magnitude of a first pulse wave amplitude indicated by an output of the first pulse wave sensor, and second indicator information indicating a magnitude of a second pulse wave amplitude indicated by an output of the second pulse wave sensor, are in positions on the display respectively corresponding to the first pulse wave sensor and the second pulse wave sensor disposed spaced apart.

2. The display control device according to claim 1 further configured to display guidance information on the display for adjusting a relative positional relationship between the sensor unit and the measurement site, in accordance with the magnitude of the first pulse wave amplitude and the magnitude of the second pulse wave amplitude.

3. The display control device according to claim 2 further configured to display the guidance information on a same screen of the display together with the first indicator information and the second indicator information.

4. The display control device according to claim 2, wherein
the guidance information includes information providing guidance related to a direction of movement for moving a position of the sensor unit relative to the measurement site.

5. The display control device according to claim 2, wherein
when the magnitude of the first pulse wave amplitude or the magnitude of the second pulse wave amplitude does not indicate a predetermined magnitude, the guidance information displays information providing guidance related to the direction of movement.

6. The display control device according to claim 5, wherein
when the magnitude of the first pulse wave amplitude and the magnitude of the second pulse wave amplitude indicate the predetermined magnitude, the guidance information includes information providing guidance related to securing a position of the sensor unit.

7. The display control device according to claim 6, wherein
when the magnitude of the first pulse wave amplitude and the magnitude of the second pulse wave amplitude indicate the predetermined magnitude, the guidance information includes information providing guidance related to securing the position of the sensor unit instead of information providing guidance related to the direction of movement.

8. The display control device according to claim 5, wherein
information indicating a magnitude of a pulse wave amplitude corresponding each of the first pulse wave amplitude and the second pulse wave amplitude has different display modes for when the magnitude of the pulse wave amplitude indicates the predetermined magnitude and when the magnitude of the pulse wave amplitude does not indicate the predetermined magnitude.

9. The display control device according to claim 2, wherein
the guidance information includes information for evaluating a state of the attachment relative to the measurement site, and
the information for evaluating indicates different evaluations when the magnitude of the first pulse wave amplitude and the magnitude of the second pulse wave amplitude indicate the predetermined magnitude, and when the magnitude of the first pulse wave amplitude or the magnitude of the second pulse wave amplitude does not indicate the predetermined magnitude.

10. The display control device according to claim 9, wherein
when the magnitude of the first pulse wave amplitude or the magnitude of the second pulse wave amplitude does not indicate the predetermined magnitude, the guidance information includes information prompting the wrapping and attaching again.

11. The display control device according to claim 9, wherein
the measurement device further includes a communication unit configured to communicate with an information processing device that is external and includes a displaying section, and is configured to transmit the guidance information to the information processing device via the communication unit to cause the displaying section to display the guidance information.

12. The display control device according to claim 2, wherein
the pulse transit time is calculated from the magnitude of the first pulse wave amplitude and the magnitude of the second pulse wave amplitude, and
the measurement device is configured to calculate blood pressure on the basis of the pulse transit time.

13. The display control device according to claim 12, further configured to display information for evaluating a state of the attachment relative to the measurement site in association with information for evaluating the blood pressure thus calculated, wherein
the guidance information includes the information for evaluating a state of the attachment.

14. The display control device according to claim 2, further configured to cause, while the guidance information is displayed, upon changing the magnitude of the first pulse wave amplitude or the magnitude of the second pulse wave amplitude, the guidance information to include information providing notification of the change.

15. A non-transitory recording medium of a program for executing a display control method of a device on a computer, the device including
a belt wrapped around and attached to a measurement site for measuring a pulse transit time,
a sensor unit provided on an inner peripheral surface of the belt that is toward the measurement site when the belt is attached, and
a display provided on an outer peripheral surface of the belt opposite to the inner peripheral surface,
the display provided on the outer peripheral surface in a portion configured to face a portion where the sensor unit being positioned when the belt is attached, and
the sensor unit including a first pulse wave sensor and a second pulse wave sensor disposed spaced apart from each other in a width direction of the belt,
the display control method comprising:
acquiring first indicator information indicating a magnitude of a first pulse wave amplitude indicated by an output of the first pulse wave sensor, and second indicator information indicating a magnitude of a second pulse wave amplitude indicated by an output of the second pulse wave sensor; and
displaying the first indicator information and the second indicator information in positions on the display respectively corresponding to the first pulse wave sensor and the second pulse wave sensor disposed spaced apart.
